(19) United States
(12) Patent Application Publication
(10) Pub. No.: US 2025/0281139 A1
(43) Pub. Date: Sep. 11, 2025

(54) SYSTEM FOR AND METHOD OF PLAQUE SCORING OF CORONARY ARTERIES

(71) Applicant: Artrya Limited, Perth (AU)

(72) Inventors: Sze Man Simon Kwok, Perth (AU); Kai Niu, Perth (AU); Girish Dwivedi, Perth (AU); Abdul Rahman Ihdayhid, Perth (AU)

(21) Appl. No.: 19/074,289

(22) Filed: Mar. 7, 2025

(30) Foreign Application Priority Data
Mar. 8, 2024 (AU) AU2024900612
Aug. 27, 2024 (AU) AU2024902678

(51) Int. Cl. A61B 6/00; A61B 6/03; A61B 6/50; G06T 7/00; G06T 7/62; G16H 50/30

(52) U.S. Cl. CPC A61B 6/5217; A61B 6/03; A61B 6/481; A61B 6/504; G06T 7/0012; G06T 7/62; G16H 50/30; G06T 2207/20024; G06T 2207/20081; G06T 2207/30048; G06T 2207/30096; G06T 2207/30101

(57) ABSTRACT

A method of automatically determining a plaque score for coronary arteries of a patient is disclosed. The method involves receiving cardiac CT data indicative of a cardiac CT scan carried out on the patient, and analysing the cardiac CT data to identify plaque volumes to be included in the plaque score, the plaque volumes located on the coronary arteries. The method also includes determining locations of the identified plaque volumes on the coronary arteries, applying machine learning to the cardiac CT data to identify a plurality of primary key points on the coronary arteries, determining a distance between each identified plaque volume and an associated primary key point, and determining a plaque score based on a heart dimension value and, for each identified plaque volume, the determined distance from the identified plaque volume to the associated primary key point.

### RELATED APPLICATIONS

This application claims the benefit under 35 U.S.C. § 119(a)-(d) of Australian Patent Application No. AU2024900612, titled “A SYSTEM FOR AND METHOD OF CALCIUM SCORING CORONARY ARTERIES” and filed on Mar. 8, 2024, and Australian Patent Application No. AU2024902678, titled “A SYSTEM FOR AND METHOD OF PLAQUE SCORING OF CORONARY ARTERIES” and filed on Aug. 27, 2024, the entire contents of which are hereby incorporated by reference as if fully set forth herein.

### TECHNICAL FIELD

The present disclosure relates to a method of and system for plaque scoring of coronary arteries.

### BACKGROUND

Coronary Artery Calcium (CAC) scores are an important indicator of Coronary Artery Disease (CAD). The calculation of CAC scores may be a semi-autonomous process that uses software to detect potential areas of calcification, but requires a trained expert to delineate between coronary artery calcification, other vessel calcification, such as aortic calcification, and other calcium containing features such as ribs or spine. CAC scores may also be determined automatically using machine learning.

The most common CAC score is calculated using Agatston's method of density weighted area calculation using gated, unenhanced CT scans.

Agatston CAC scores provide a score that is indicative of the overall calcified plaque, but the score does not provide any indication of the pattern of calcified plaque in a patient. For example, 2 patients may have very similar Agatston CAC scores but very different calcium distributions within the coronary artery tree to the extent that a patient with highly diffuse calcified plaque will typically be associated with a much worse prognosis compared to a patient with a more concentrated calcified plaque distribution.

Agatston CAC scores also do not take into account the presence and significance of non-calcified plaque.

In the context of coronary CT angiography, a coronary artery disease reporting and data system score (CAD-RADS) is often used to classify severity of coronary artery disease (CAD). With the CAD-RADS scoring system, the following categories apply:
CAD-RADS 0: No visible coronary artery disease
CAD-RADS 1-2: Minimal CAD (<49%)
CAD-RADS 3: Moderate stenosis (50-69%)
CAD-RADS 4 Severe stenosis (70-99%)
CAD-RADS 5: Complete occlusion (100%)

However, as with Agatston scores, CAD-RADS scores are a measure of calcified plaque only and do not provide an indication of how diffuse or concentrated the plaque is.

An alternative arrangement for assessing coronary artery plaque and the associated risk of adverse cardiovascular events is desired.

### SUMMARY

In accordance with a first aspect of the present disclosure, there is provided a method of automatically determining a plaque score for coronary arteries of a patient, the method comprising:
receiving cardiac CT data indicative of a cardiac CT scan carried out on the patient;
analysing the cardiac CT data to identify plaque volumes to be included in the plaque score, the plaque volumes located on the coronary arteries;
determining locations of the identified plaque volumes on the coronary arteries;
applying machine learning to the cardiac CT data to identify a plurality of primary key points on the coronary arteries;
determining a distance between each identified plaque volume and an associated primary key point; and
determining a plaque score based on a heart dimension value and, for each identified plaque volume, the determined distance from the identified plaque volume to the associated primary key point.

In an embodiment, the locations of the identified plaque volumes on the coronary arteries are determined using machine learning.

In an embodiment, the key points include at least one primary key point corresponding to an ostium location at which a coronary artery connects to a patient's aorta.

In an embodiment, the key points include 2 primary key points corresponding to 2 ostium locations at which two respective coronary arteries connect to a patient's aorta.

In an embodiment, the 2 primary key points correspond to a first ostium location at which a RCA coronary artery connects to the patient's aorta, and a second ostium location at which a LM coronary artery connects to the patient's aorta.

In an embodiment, the key points include at least one secondary key point corresponding to a location on a coronary artery associated with a coronary artery branch location.

In an embodiment, the at least one secondary key point corresponds to a location on the LM coronary artery of the patient at which the LM coronary artery branches to a LCx coronary artery and a LAD coronary artery.

In an embodiment, if an identified plaque volume is on a coronary artery that connects directly to the aorta at an ostium, then the method comprises determining the distance by determining a straight line distance from the plaque volume to a primary key point associated with the ostium.

In an embodiment, if an identified plaque volume is on a coronary artery that connects indirectly to the aorta at an ostium through a branch, then the method comprises determining the distance by determining a straight line distance from the plaque volume to a secondary key point associated with the branch plus a straight line distance from the secondary key point to a primary key point associated with the ostium.

In an embodiment, the distance is determined from a centroid of an identified plaque volume.

In an embodiment, the heart dimension value includes a maximum heart diameter value.

In an embodiment, the plaque score is determined based on volume magnitude of the plaque volumes.

In an embodiment, the cardiac CT data is non-contrast CT data and the plaque volumes are calcified volumes.

In an embodiment, the method comprises segmenting an ascending or descending aorta of the patient from the cardiac non-contrast CT data, and using the segmented ascending and descending aorta to remove from consideration plaque volumes considered to be present on the ascending or descending aorta and therefore not on the coronary arteries.

In an embodiment, the method comprises segmenting a cardiac region from the cardiac non-contrast CT data, and using the segmented cardiac region to remove from consideration plaque volumes that are considered to be outside the heart region.

In an embodiment, the plaque score for calcified plaque volumes is determined based on a non-contrast derived CT volume and using the following equation:

$$D = {\sum }_{i = 1}^{N} {s}_{i} {w}_{i} \frac{H}{{d}_{i}}$$

where N is the total number of calcified plaque volumes with Hounsfield Units (HU) >=130; s is the volume magnitude of each calcified plaque volume in mm³; w is a weighting factor of the calcified plaque volume's maximum attenuation; d is the straight-line distance from the calcified plaque volume's centroid to the ostium (via the bifurcation point if the lesion is located beyond the left main artery) in mm; and H is the maximum heart diameter measured along a short axis of the segmented heart region in an axial view in mm.

In an embodiment, the weighting factor w is given by:

$$w ( {X}_{i} ) = \{ \begin{matrix}0 & if max ( {X}_{i} ) < 130 \\ 1 & if 130 <= max ( {X}_{i} ) <= 199 \\ 2 & if 200 <= max ( {X}_{i} ) <= 299 \\ 3 & if 300 <= max ( {X}_{i} ) <= 399 \\ 4 & if 400 <= max ( {X}_{i} ) <= 999 \\ 1 & if 1000 <= max ( {X}_{i} )\end{matrix}$$

In an embodiment, the method includes filtering CT data using defined filtering characteristics to identify the plaque volumes to be included in the plaque score.

In an embodiment, the defined filtering characteristics include HU values.

In an embodiment, the cardiac CT data is contrast CT data and the plaque volumes include non-calcified volumes.

In an embodiment, the defined filtering characteristics include plaque volume characteristics.

In an embodiment, the defined filtering characteristics include plaque composition.

In an embodiment, the plaque score is determined based on a stenosis severity parameter.

In an embodiment, the plaque score is determined based on a number of high risk plaque features.

In an embodiment, a plurality of high risk plaque features are defined and each high risk plaque feature has an associated scaling factor. The high risk plaque features may include positive remodelling, low attenuation plaque, napkin ring sign and/or spotty calcification.

In an embodiment, the plaque score for a contrast derived CT volume may be determined using the following equation when values for plaque volume magnitudes are known:

$$D = {\sum }_{i = 1}^{N} ( {s}_{i} + {p}_{i} ) \frac{V}{{d}_{i}} {\sum }_{j = 1}^{3} {α}_{i} {v}_{i , j}$$

where N is the total number of plaque volumes; s is stenosis percentage/100; p is the number of high risk plaque features; V is the left ventricle volume magnitude in mm³; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque.

In an alternative embodiment, the plaque score for a contrast derived CT volume may be determined using the following equation when values for plaque volume magnitudes are known:

$$D = {\sum }_{i = 1}^{N} ( {s}_{i} + {p}_{i} ) \frac{V}{{d}_{i}} {\sum }_{j = 1}^{3} {α}_{i} {v}_{i , j}$$

where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque.

In an embodiment, the plaque score for a contrast derived CT volume may be determined using the following equation when values for plaque volume magnitudes are not known or not all known:

$$D = {\sum }_{i = 1}^{N} ( {s}_{i} + {p}_{i} ) \frac{V}{{d}_{i}}$$

where N is the total number of plaque volumes; s is stenosis percentage/100; p is the number of high risk plaque features; V is the left ventricle volume magnitude in mm³; and d is the straight-line distance from the plaque volume's centroid to the ostium (via the bifurcation point if the lesion is located beyond the left main artery) in mm.

In an alternative embodiment, the plaque score for a contrast derived CT volume may be determined using the following equation when values for plaque volume magnitudes are not known or not all known:

$$D = {\sum }_{i = 1}^{N} ( {s}_{i} + {p}_{i} ) \frac{V}{{d}_{i}}$$

where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; and d is the straight-line distance from the plaque volume's centroid to the ostium (via the bifurcation point if the lesion is located beyond the left main artery) in mm.

In accordance with a second aspect of the present disclosure, there is provided a system for automatically determining a plaque score for coronary arteries of a patient, the system comprising:
a plaque volume identifier arranged to receive cardiac CT data indicative of a cardiac CT scan carried out on a patient, analyse the cardiac CT data to identify plaque volumes located on the coronary arteries to be included in the plaque score, and determine locations of the identified plaque volumes on the coronary arteries;
a key point determiner arranged to apply machine learning to the cardiac CT data to identify a plurality of primary key points on the coronary arteries;
a key point distance determiner arranged to determine a distance between each identified plaque volume and an associated primary key point; and
a plaque score calculator arranged to determine a plaque score based on a heart dimension value and, for each identified plaque volume, the determined distance from the identified plaque volume to the associated primary key point.

### BRIEF DESCRIPTION OF THE DRAWINGS

Example embodiments will now be described, by way of example only, with reference to the accompanying drawings, in which:

FIG. 1 is a schematic block diagram of a system for scoring plaque using cardiac non-contrast CT data according to an embodiment;

FIG. 2 is a flow diagram illustrating a method of scoring plaque according to the embodiment shown in FIG. 1;

FIG. 3 is a diagrammatic representation of an example determined distance between an identified plaque component on an RCA coronary artery and an associated primary key point;

FIG. 4 is a diagrammatic representation of a further example determined distance between an identified plaque component on an LM coronary artery and an associated primary key point;

FIG. 5 is a diagrammatic representation of a further example determined distance between an identified plaque component on a LCx coronary artery and an associated primary key point via a secondary key point;

FIG. 6 is a diagrammatic representation of a further example determined distance between an identified plaque component on a LAD coronary artery and an associated primary key point via a secondary key point;

FIG. 7 is a schematic block diagram of a system for scoring plaque using cardiac contrast CT data according to an embodiment;

FIG. 8 is an example transverse cross-sectional view of a coronary artery vessel;

FIG. 9 is a schematic block diagram of a plaque score determiner of the system shown in FIG. 7; and

FIG. 10 is a flow diagram illustrating a method of scoring coronary artery plaque according to an embodiment, the coronary plaque identified using cardiac contrast CT data.

### DESCRIPTION OF CERTAIN EMBODIMENTS

The present disclosure relates to an automated method for determining a coronary artery score that is indicative of the presence, extent and distribution of plaque on a patient's coronary arteries. The plaque score may be based on calcified plaque, non-calcified plaque or a mixture of calcified and non-calcified plaque, and the type of plaque included in the plaque score may be customised by a user.

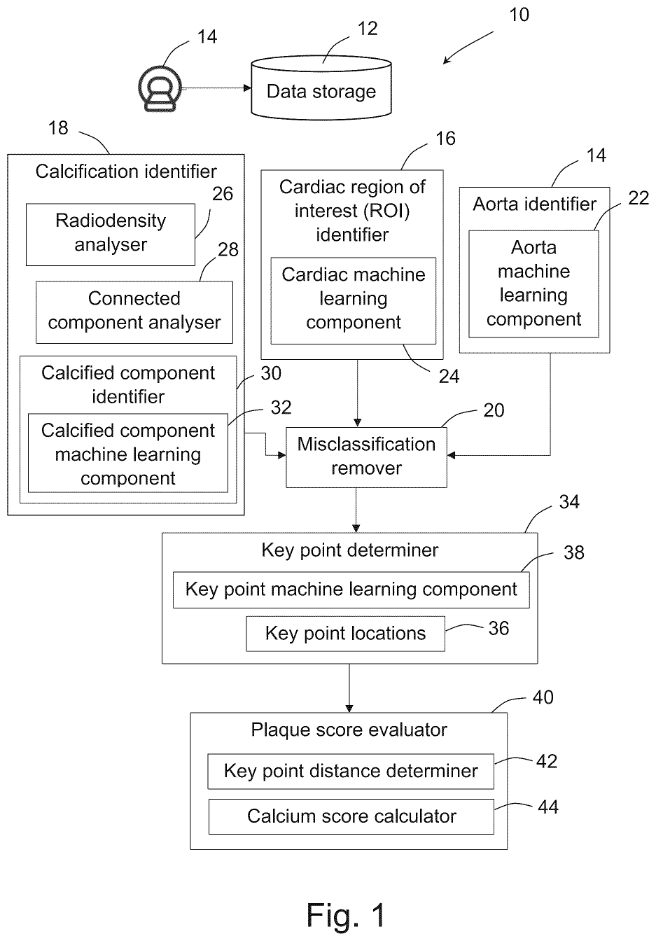

Referring to FIG. 1 of the drawings, an example system 10 for plaque scoring of coronary arteries is shown. In this example, the plaque score is based on calcified plaque and, as such, the score is obtained using gated, non-contrast cardiac computed tomography (CT) scans.

The system 10 includes a data storage device 12 arranged to store cardiac non-contrast CT data received from a CT scanning device 14, an aorta identifier 14 for identifying ascending and descending aorta components in the cardiac non-contrast CT data, a cardiac identifier 16 for identifying a cardiac region in the non-contrast CT data, a calcification identifier 18 for identifying calcified components in the cardiac non-contrast CT data, and a misclassification remover 20 that uses the information from the aorta identifier 14 and the cardiac identifier 16 to remove calcified volumes from consideration that are not considered to be associated with a coronary artery.

In this example, the aorta identifier 14 includes an aorta machine learning component 22 arranged to predict using machine learning whether each voxel in a received patient cardiac non-contrast CT data volume is part of the ascending or descending aorta of the patient.

The segmented ascending and descending aorta is subsequently used to remove from consideration calcifications present on the ascending or descending aorta and therefore not on the coronary arteries.

In this example, the cardiac identifier 16 includes a cardiac machine learning component 24 arranged to predict using machine learning voxels in received patient cardiac non-contrast CT data volume that are part of a cardiac region of the patient.

The segmented cardiac region is subsequently used to remove from consideration calcifications that are considered to be outside the heart region.

In this example, the calcification identifier 18 includes a radiodensity analyser 26 arranged to identify candidate voxels associated with calcified components, for example by applying a Hounsfield Unit thresholder to the voxel data so that only voxels with an associated radiodensity above a defined level are passed. In the present example, the defined HU level is 130, although any other suitable HU level is envisaged.

The calcification identifier 18 also includes a connected component analyser 28 arranged to use a connected component technique to identify neighbouring voxels that belong to the same component and therefore form part of a candidate calcified component.

The calcification identifier 18 also includes a calcified component identifier 30 arranged to predict whether a candidate calcified component is located on a coronary artery, and if so to predict the coronary artery on which the candidate calcified component is located and the location of the candidate calcified component. The calcified component identifier 30 may achieve this using a calcified component machine learning component 32.

In the present example, each of the aorta, cardiac and calcified component machine learning components 22, 24, 32 uses machine learning that is trained using a sufficient number of relevant, known outcome, non-contrast CT scans. For example, the machine learning components 22, 24, 32 may each use a convolutional neural network (CNN). It will be appreciated that various suitable machine learning arrangements are envisaged, for example a wide variety of convolutional neural networks (CNN) can be effectively employed for semantic segmentation. In medical applications, the Unet and Vnet type CNN architectures are commonly used.

In order to train the aorta machine learning component 22, non-contrast CT scan data covering the coronary region of a plurality of patients is received and CT scan images of the ascending and descending aorta components are annotated by experts so that a library of ground truth training data is produced. The ground truth aorta training data derived from each scan constitutes a map of voxels identified as being part of the ascending or descending aorta, and the aorta machine learning component 22 is trained using the aorta maps to recognise components of a CT scan that are part of the ascending or descending aorta.

Similarly, in order to train the cardiac machine learning component 24, cardiac non-contrast CT data covering the coronary region of a plurality of patients is received and the CT scan images of the heart region are annotated by experts so that a library of ground truth training data is produced. The ground truth cardiac training data derived from each scan constitutes a map of voxels identified as being part of the cardiac region, and the cardiac machine learning component is trained using the cardiac maps to recognise components of a CT scan that are part of the cardiac region.

The predicted candidate calcifications produced by the calcification identifier 18 are cross-checked against the predicted ascending and descending aorta information and the predicted cardiac area information by the misclassification remover 20, and any candidate calcifications that are considered to relate to noise, or to be present on the ascending or descending aorta, or located outside the cardiac area, are removed.

A plaque score is determined based on plaque volumes considered to be present on the coronary arteries, in the present example calcified plaque component volumes. The plaque score also incorporates a heart dimension value and, for each identified plaque volume, the determined distance from the identified plaque volume to an associated primary key point defined on the coronary arteries, in this example located at interfaces between the RCA and LM coronary arteries and the aorta.

In the present example, the plaque score is determined using the following formula:

$$D = {\sum }_{i = 1}^{N} {s}_{i} {w}_{i} \frac{H}{{d}_{i}} \qquad ( 1 )$$

where N is the total number of plaque volumes with Hounsfield Units (HU)>=130; s is the volume magnitude of each plaque volume in mm³; w is a weighting factor of the plaque volume's maximum attenuation; d is the straight-line distance from the plaque volume's centroid to the ostium (via the bifurcation point if the lesion is located beyond the left main artery) in mm; and H is the maximum heart diameter measured along a short axis of the segmented heart region in an axial view in mm,
and where

$$w ( {X}_{i} ) = \{ \begin{matrix}0 & if max ( {X}_{i} ) < 130 \\ 1 & if 130 <= max ( {X}_{i} ) <= 199 \\ 2 & if 200 <= max ( {X}_{i} ) <= 299 \\ 3 & if 300 <= max ( {X}_{i} ) <= 399 \\ 4 & if 400 <= max ( {X}_{i} ) <= 999 \\ 1 & if 1000 <= max ( {X}_{i} )\end{matrix}$$

In the present example, the distance d is the distance from a centroid of a plaque volume to a defined key point on the coronary arteries that corresponds to an ostium at which a coronary artery connects to the aorta. In the present example, a plurality of key points are defined that include primary key points respectively associated with a point on the coronary arteries representing an ostium, and secondary key points that correspond to locations on the coronary arteries associated with artery bifurcation locations.

The weighting factor w is similar to the weighting factor used to calculate an Agatston score, but with the present arrangement, if the HU value is greater than 1000, w is set to 1.

It will be understood that the present coronary artery plaque score, calculated according to the above formula, incorporates spatial distribution of plaque volumes into the score by incorporating measured plaque volume distances from coronary artery vessel origins. In this way, an indication of plaque distribution in a patient's coronary arteries is suggested from the score.

As shown in FIG. 1, the system 10 includes a key point determiner 34 arranged to identify a plurality of key point locations 36 on coronary arteries of a patient, in this example the plurality of key points including a plurality of primary key points associated with ostium locations and at least one secondary key point associated with one or more bifurcation locations. In the present embodiment, a first primary key point corresponds to an ostium associated with a right coronary artery (RCA), a second primary key point corresponds to an ostium associated with a left main artery (LM), and a secondary key point corresponds to a bifurcation location wherein the left main artery (LM) branches to a left circumflex artery (LCx) and a left anterior descending artery (LAD).

The key point determiner 34 includes a key point machine learning component 38 arranged to predict using machine learning the locations of the first and second primary key points and the secondary key point in a CT volume associated with a non-contrast CT scan.

In the present example, the key point machine learning component 38 comprises a convolutional neural network (CNN) that includes 3D convolution layers and is trained to predict the key points in a received CT volume. The convolutional neural network in this example is trained using annotations of key points in CT volumes.

As shown in FIG. 1, the system 10 also includes a plaque score evaluator 40 that includes a key point distance determiner 42 arranged to determine, for each identified coronary artery plaque volume, a distance between a centroid of the plaque volume and a defined primary key point on the relevant coronary artery.

The arrangement is such that:
if the plaque volume is on a coronary artery that connects directly to the aorta at an ostium, then the distance d corresponds to a straight line distance from the plaque volume centroid to the primary key point associated with the ostium; and
if the plaque volume is on a coronary artery that connects indirectly to the aorta through a branch, then the distance d corresponds to a straight line distance from the plaque volume centroid to a secondary key point associated with the branch location plus a straight line distance from the secondary key point to the primary key point associated with the ostium.

In the present example, the system is configured to identify the 4 main defined coronary arteries, that is the RCA, LM, LCx and LAD coronary arteries.

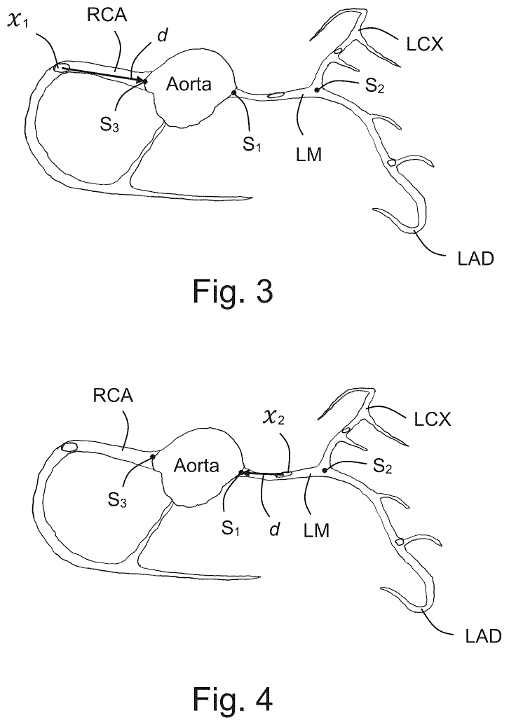

Examples of distances d associated with determined coronary artery plaque volumes are shown in FIGS. 3 to 6. A first primary key point S₁ corresponds to an ostium associated with connection of the LM vessel to the aorta, a second primary key point S₃ corresponds to an ostium associated with connection of the RCA vessel to the aorta, and a secondary key point S₂ corresponds to a branch location at which the LM vessel bifurcates into the LCx vessel and the LAD vessel.

As shown in FIG. 3, a plaque volume x₁ is present on the RCA coronary artery. The distance d determined by the key point distance determiner 42 is the straight line distance from the centroid of plaque volume x₁ to the second primary key point S₃.

As shown in FIG. 4, a plaque volume x₂ is present on the LM coronary artery. The distance d determined by the key point distance determiner 42 is the straight line distance from the centroid of plaque volume x₂ to the first primary key point S₁.

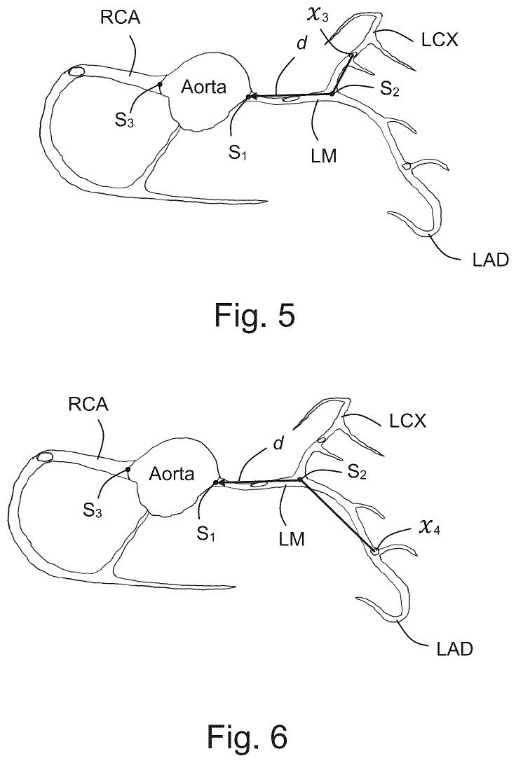

As shown in FIG. 5, a plaque volume x₃ is present on the LCx coronary artery. The distance d determined by the key point distance determiner 42 is the straight line distance from the centroid of plaque volume x₃ to the secondary key point S₂, plus the straight line distance from the secondary key point S₂ to the first primary key point S₁.

As shown in FIG. 6, a plaque volume x₄ is present on the LAD coronary artery. The distance d determined by the key point distance determiner 42 is the straight line distance from the centroid of plaque volume x₄ to the secondary key point S₂, plus the straight line distance from the secondary key point S₂ to the first primary key point S₁.

The plaque score evaluator 40 also includes a plaque score calculator 44 arranged to calculate the plaque score according to the above equation (1).

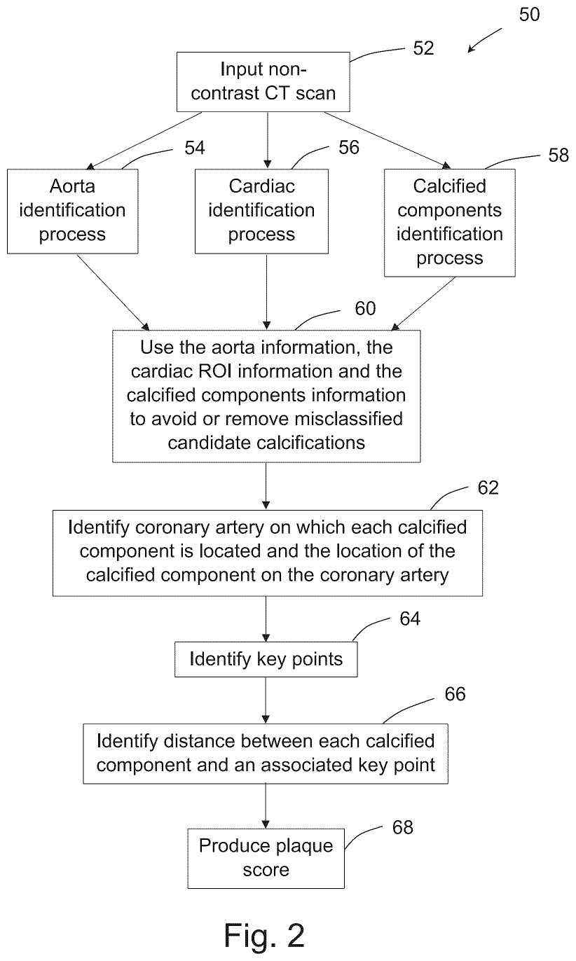

Referring to FIG. 2, a flow diagram 50 illustrating an example method of plaque scoring using the system 10 is shown, in this example the method producing a plaque score based on calcified plaque volumes.

After receiving a non-contrast scan CT volume, as indicated at step 52, an aorta identification process is carried out to segment the patient's aorta in the CT volume and a cardiac identification process is carried out to segment a patient's heart region in the CT volume, as indicated at steps 54 and 56. Since the present embodiment relates to a plaque score based on calcified plaque only, a calcified components identification process is also carried out to identify calcified components in the CT volume that are considered to be located on the coronary arteries, as indicated at step 58.

As indicated at step 60, a misclassification removal process is then implemented using the identified calcified components, the segmented aorta and the segments heart region used to remove calcified components from consideration that are considered to be disposed on the aorta or outside of the heart region.

After identification of the coronary artery on which each calcified component is located and the location of the calcified component on the coronary artery, as indicated at step 62, a plurality of defined key points are identified on the coronary arteries using machine learning, the key points including primary key points associated with ostium locations and at least one secondary key point associated with a branch location, as indicated at step 64. The key point distance determiner 42 is then used to produce values for the distance between each calcified volume and the relevant ostium location, as indicated at step 66, and a plaque score is produced using the above equation (1), as indicated at step 68.

While the above embodiment is described in relation to a plaque score based only on calcified volumes identified using a non-contrast CT scan and considered to be disposed on a patient's coronary arteries, it will be understood that the disclosure has broader application to production of a plaque score for other types of plaque or a combination of plaque types.

For example, the present system may be arranged to produce a plaque score based on a contrast CT scan and a disease assessment that also identifies non-calcified plaque volumes. With this arrangement, the system may enable a user to apply a plaque filter to identified plaque volumes so that the plaque score provides an indication of the distribution of one or more specific plaque types on a patient's coronary arteries.

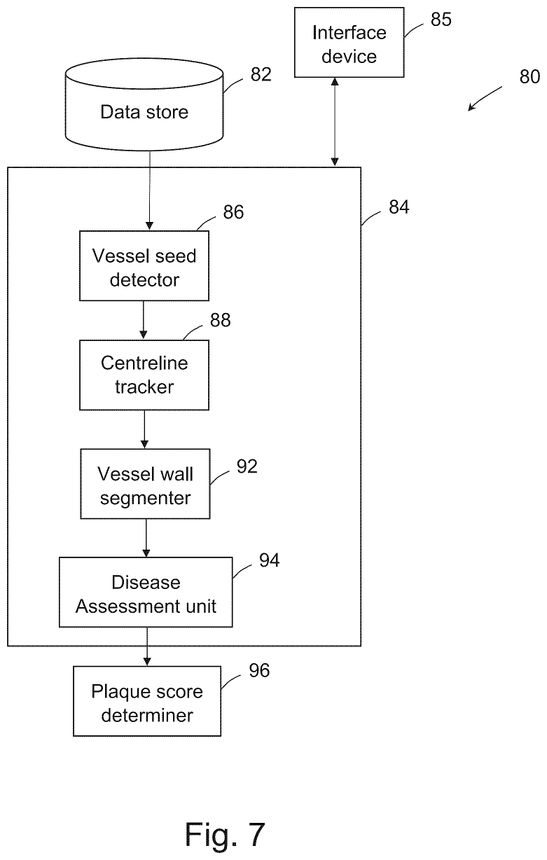

In an embodiment shown in FIGS. 7 to 16, a system 80 for plaque scoring of coronary arteries is shown wherein the plaque volumes to form the basis of the plaque score may include calcified plaque, non-calcified plaque and/or mixed plaque.

In order to produce a plaque score that is indicative of a desired type or types of plaque, the system 80 may be arranged to facilitate filtering of identified coronary artery plaque volumes, for example using Hounsfield Unit filtering to specify plaque type based on density. Alternatively, or in addition, plaque filtering techniques may be based on determined plaque composition characteristics, such as ‘high risk’ plaque type.

‘High-risk’ plaque features, sometimes referred to a ‘vulnerable plaque’ features, include spotty calcification, low attenuation plaque, positive remodelling, and a feature referred to as a ‘napkin ring sign’.

For example, non-calcified low attenuation plaques are characterised by Hounsfield Unit (HU) values in the range −30 to 30.

In a further example, spotty calcification is defined as a relatively small calcification surrounded by non-calcified or mixed plaque. To detect spotty calcification, voxels that are predicted to be associated with calcified plaques are initially determined, for example by filtering using a defined radiodensity measure, such as a Hounsfield Unit (HU) value greater than 350. Surrounding areas of the calcified spots are then analysed to determine whether the voxels surrounding the identified calcified spots have HU values consistent with non-calcified or mixed plaques. Spotty calcifications are also characterised as being smaller than 3 mm in diameter, and accordingly a size threshold may also be applied to identify them.

The system 80 is arranged to identify plaque present on coronary arteries using contrast coronary computed tomography (CT) data.

Referring to FIG. 7, received contrast CT image data is stored in a data storage device 82 that may include one or more databases.

The system 80 includes an analysis device 84 in communication with the data storage device 82, and arranged to analyse contrast CT image data stored in the data storage device 82 and identify plaque volumes present on a patient's coronary arteries and for example stenosis levels on the coronary arteries. An example analysis device 84 is described more particularly in applicants international patent application No. WO2022/221921, the contents of which are hereby incorporated by reference.

The system 80 may be arranged to facilitate access by a user, for example using a suitable network enabled interface device 85 in any suitable way.

Using the interface device 85, a user is able to instigate analysis and/or view the results of analysis of CT data stored at the data storage device 82. During analysis, the analysis device 84 extracts relevant CT data from the data storage device 82 and carries out analysis processes on the CT data in order to identify plaque volumes present on a patient's coronary arteries.

A user interacts with the system 80 using a user interface that communicates patient coronary artery disease related information to the user and facilitates reception of instructions and/or information from the user, for example relating to the type of plaque to be included in the plaque score.

In the present example, the system 80 is also arranged to use machine learning to detect and track coronary artery centrelines, to use machine learning to estimate the location of inner and outer walls of coronary arteries based on the centrelines, and to identify plaque volumes on coronary arteries using the estimated inner and outer walls together with an analysis of the composition and characteristics of identified gaps between the inner and outer walls.

However, it will be understood that other methodologies are envisaged for analysing coronary arteries for presence of plaque volumes.

In this example, the analysis component 84 relies on segmentation of inner and outer walls of the coronary arteries and the information produced by this is used to detect and assess the disease burden in the scan. In order to accurately segment the vessel walls, centrelines of the coronary arteries are first determined by identifying a plurality of seed points on each centreline that are likely to be located on a centreline of a coronary artery. To facilitate this process, a contrast agent is injected into the blood stream to increase contrast and in this example increase a Hounsfield Unit (HU) value of the coronary arteries compared to the surrounding tissue.

The analysis component 84 identifies for each vessel a set of predicted seed points using a vessel seed detector 86 that in this example uses multiscale filtering and supervised machine learning. In this example, a volumetric convolutional neural network (CNN) is used that is trained using ground truth data indicative of a sufficient number of example coronary artery centrelines.

The vessel seed detector 86 selects candidate seed points from the set of predicted seed points that are to form the basis of coronary artery centreline prediction. The candidate vessel seed points are determined from the set of predicted seed points based on one or more defined constraints, such as seed points that have a radiodensity value, such as a Hounsfield Unit (HU) value, above a defined amount, or a defined number of seed points above a defined HU threshold, such as a defined number of seed points that have the highest HU values. In one example, the candidate vessel seed points that have a HU value between 100 and 600 are selected as candidate seed points.

A centreline tracker 88 then considers the determined candidate seed points and predicts from an instant seed point the most probable direction to the next seed point on the coronary artery in three dimensional space using machine learning, and in this way vessel centreline seed points are identified that are likely to lie on the currently considered coronary artery. In this example, the centreline tracking process starts at a predicted seed point located at an endmost location on an artery centreline. The candidate seed points identified in this way as located on a coronary artery centreline are connected together so as to define a complete coronary artery.

The centreline tracker 88 is arranged to detect the four main coronary arteries first—the Left Main (LM), Left Anterior Descending (LAD), Left Circumflex (LCX) and the Right Coronary Artery (RCA), then after the main coronary arteries have been detected, branches on the primary coronary arteries are detected that were not initially identified as viable centrelines.

The centreline tracker 88 examines the HU values perpendicular to the centreline direction of a vessel, and estimates the approximate radius of the vessel by finding the boundary of the coronary artery based on the HU value, since the HU value decreases significantly outside of the vessel wall. Once the boundary has been located on each side of the centreline, the vessel's diameter can be measured.

Branches are detected based on the rate of change in measured diameter of the vessel along the length of a centreline. For example, if the measured diameter of the vessel increases by more than 10% along the centreline, then decreases back to its original size it is marked as a detected branch, noting that coronary vessels naturally decrease in size from a proximal to a distal location. At the coronary ostia, vessels may have a diameter of about 4 mm, whilst at a distal location the vessel diameter typically reduces to less than 1 mm. Branch detection is therefore carried out based on the rate of change of the estimated diameter to detect points along the centreline from which another coronary artery is branching.

The analysis component 84 may then attach semantically meaningful labels to the tracked artery centrelines, for example using machine learning, so that clinicians can more easily identify the vessels.

In this example, the analysis component 84 is also arranged to improve the reliability of the centreline tracking process by facilitating reconfiguration of the vessel seed detector 86 if the analysis carried out by the centreline tracker 88 is incorrect or incomplete, for example because the vessel seed detector 86 has generated too many or insufficient seed points. For example, this may be achieved by lowering the constraint applied by the vessel seed detector 86 so that more candidate vessel seed points are produced, thereby increasing the probability of detecting the vessel in a subsequent iteration.

After all desired coronary arteries have been satisfactorily tracked and labelled, a vessel wall segmenter 92 uses the tracked centrelines to analyse the CT data associated with the coronary arteries, in particular to carry out an inner and outer vessel wall segmentation process.

The vessel wall segmenter 92 uses a machine learning component to produce inner and outer wall lumen masks that can then be used to identify coronary artery disease associated with the presence of calcified and non-calcified plaques. In this example, the machine learning component is a supervised volumetric convolutional neural network (CNN) that is trained using ground truth training data indicative of a sufficient number of example transverse coronary artery image slices, in this example image slices that are perpendicular to and intersecting with the artery centrelines. The training data in this example includes inner and outer artery walls and relevant imaging artefacts that have been annotated by medical experts, and covers a wide range of examples of different coronary vessels with varying degrees of disease.

It will be understood that after completion of coronary artery wall segmentation, the system has sufficient data to define the inner and outer vessel wall configurations of the detected coronary arteries. Using this data, it is possible to determine the presence of plaque volumes by analysing voxels associated with gap regions between the inner and outer vessel walls, and make determinations, for example in relation to stenosis and/or presence of high risk plaque.

The analysis device 84 includes a disease assessment unit 94 arranged to identify presence of plaque volumes, including calcified, mixed and/or non-calcified plaque volumes, for example based on the characteristics, composition and/or Hounsfield Unit values of gaps in the vessel walls.

Low attenuation plaques are characterised by Hounsfield Unit (HU) values in the range −30 to 30 Hounsfield units, and therefore may be directly detected through analysis and thresholding of Hounsfield units.

A spotty calcification is defined as a relatively small calcification surrounded by non-calcified or mixed plaque. To detect spotty calcification, the disease assessment unit 84 initially determines voxels that are predicted to be associated with calcified plaques in the determined disease region between the inner and outer artery wall, for example by filtering using a defined radiodensity measure, such as a Hounsfield Unit (HU) value greater than 350. Related voxels are then associated together as calcified volumes. Spotty calcifications are characterised as being smaller than 3 mm in diameter. Non-calcified/mixed plaque is used to determine whether the voxels surrounding the identified spotty calcifications have HU values consistent with non-calcified or mixed plaques.

Positive remodelling refers to a vessel feature wherein the ratio of outer vessel diameter at the site of plaque divided by the average outer diameter of the proximal and distal vessel greater than 1.1, or Av/[(Ap+Ad)/2]>1.1.

A ‘napkin ring sign’ feature is defined in a non-calcified plaque cross-sectional image by the presence of two features—a central area of low attenuation plaque that appears to be in contact with the lumen, and a ring-like peripheral rim of higher radiodensity surrounding the central area.

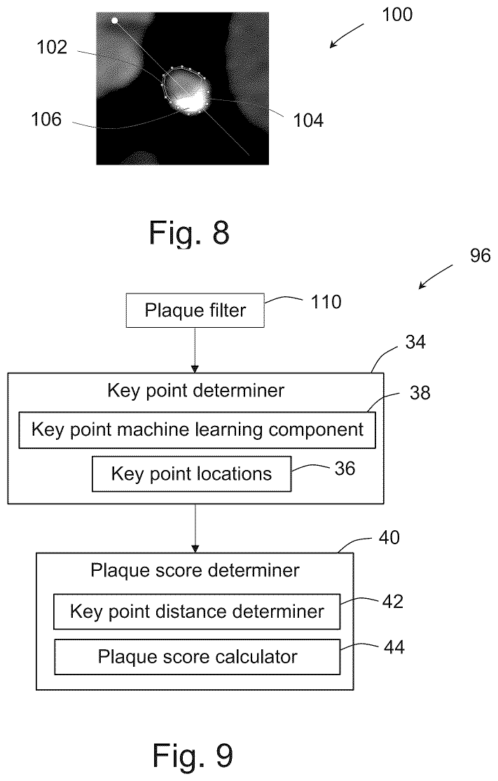

The identified plaque volumes may be displayed on the user interface device 85, for example on a 3D model of the patient's coronary arteries. The identified plaque volumes may also be viewed on the user interface device 85 by enabling a user to select a location on a coronary artery and displaying a cross section of the coronary artery at the selected location. An example artery cross-sectional view 100 is shown in FIG. 8. As shown, the cross-sectional view 100 includes segmented inner 102 and outer 104 arterial walls and an identified plaque volume 106 between the inner and outer walls 102, 104.

Referring to FIG. 9, components of the plaque score determiner 96 are shown. The plaque score determiner 96 includes a plaque filter 110 that enables a user to select one or more specific types of plaque volumes that are to form the basis of plaque score determination. In this example, the plaque filter 110 is arranged to facilitate selection of plaque volume type based on HU filtering and/or based on defined plaque characteristics, such as one or more characteristics associated with high risk plaque types.

The plaque score determiner 96 also includes a key point determiner 34 and a plaque score evaluator 40 that carry out the same functions as the key point determiner 34 and the plaque score evaluator 40 shown in FIG. 1 and described above in relation to FIGS. 3 to 6. Like and similar features are indicated with like reference numerals. As with the plaque scoring system 10 shown in FIG. 1, the key point determiner 34 is arranged to identify a plurality of key points 36 on coronary arteries of a patient, in this example the plurality of key points including a plurality of primary key points associated with ostium locations and at least one secondary key point associated with one or more bifurcation locations, and the plaque score evaluator 40 arranged to determine, for each identified plaque volume, a distance between a centroid of the plaque volume and a defined primary key point on the relevant coronary artery, and to determine a plaque score for the selected plaque volume type(s).

The plaque score may be determined using the above equation (1) if the plaque score relates to calcified plaque only. Otherwise, an alternative equation may be used.

For example, for a contrast-derived CT volume that includes non-calcified and/or mixed plaque, the plaque score may be determined using the following equation:

$$D = {\sum }_{i = 1}^{N} ( {s}_{i} + {p}_{i} ) \frac{V}{{d}_{i}} {\sum }_{j = 1}^{3} {α}_{i} {v}_{i , j} \qquad ( 2 )$$

where N is the total number of plaque volumes; s is stenosis percentage/100; p is the number of high risk plaque features; V is the left ventricle volume magnitude in mm³; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque.

It will be understood that the plaque score produced by equation 2 incorporates values for plaque volume magnitude, for example determined by the disease assessment unit 94.

In an alternative embodiment wherein plaque volume values are not available, or not all plaque volume values are available, the plaque score may be determined from a contrast determined CT volume using the following equation.

$$D = {\sum }_{i = 1}^{N} ( {s}_{i} + {p}_{i} ) \frac{V}{{d}_{i}} \qquad ( 3 )$$

where N is the total number of plaque volumes; s is stenosis percentage/100; p is the number of high risk plaque features; V is the left ventricle volume magnitude in mL; and d is the straight-line distance from the plaque volume's centroid to the ostium (via the bifurcation point if the lesion is located beyond the left main artery) in mm.

In a further alternative embodiment for use with a contrast determined CT volume, the plaque score may be determined using the following equation.

$$D = {\sum }_{i = 1}^{N} ( {s}_{i} + {p}_{i} ) \frac{V}{{d}_{i}} {\sum }_{j = 1}^{3} {α}_{j} {v}_{i , j} \qquad ( 4 )$$

where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque.

Similarly with equation 2 and as an alternative to equation 2, it will be understood that the plaque score produced by equation 4 incorporates values for plaque volume, for example determined by the disease assessment unit 94.

Similarly with equation 4 and as an alternative to equation 4 when plaque volume values are not available, or not all plaque volume values are available, the plaque score may be determined using the following equation.

$$D = {\sum }_{i = 1}^{N} ( {s}_{i} + {p}_{i} ) \frac{V}{{d}_{i}} \qquad ( 5 )$$

where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; and d is the straight-line distance from the plaque volume's centroid to the ostium (via the bifurcation point if the lesion is located beyond the left main artery) in mm.

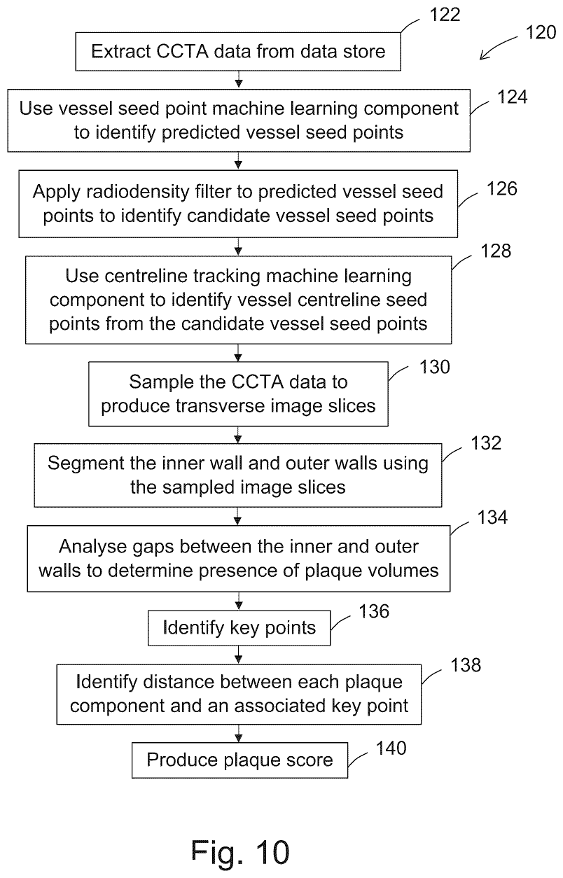

A flow diagram 120 illustrating steps 122 to 140 of an example method of plaque scoring using the system shown in FIG. 7 is shown in FIG. 10.

As indicated at step 122, contrast CCTA associated with a patient is first extracted from the data store 82, then predicted centreline seed points are obtained from the CCTA data, in this example using a trained centreline seed point machine learning component, as indicated at step 124. Candidate centreline seed points are then obtained by radiodensity filtering using Hounsfield Unit (HU) values above a defined amount, or a defined number of seed points above a defined HU threshold, such as a defined number of seed points that have the highest HU values, as indicated at step 126.

As indicated at step 128, a centreline tracker 88 then considers the determined candidate seed points and predicts from an instant seed point the most probable direction to the next seed point on the coronary artery in three dimensional space using machine learning, and in this way vessel centreline seed points are identified that are likely to lie on a centreline of the currently considered coronary artery.

As indicated at step at steps 130 and 132, after centrelines of all desired coronary arteries have been determined, the centrelines are used to obtain transverse images of the vessels, and the transverse images are analysed to segment inner and outer vessel walls 102, 104. After segmenting the vessel inner and outer walls, gaps between the inner and outer walls 102, 104 are analysed to determine the presence of plaque volumes, the type of plaque volumes present and the locations of the plaque volumes, as indicated at step 134.

A plurality of defined key points are identified on the coronary arteries using machine learning, the key points including primary key points associated with ostium locations and at least one secondary key point associated with a branch location, as indicated at step 136. The key point distance determiner 42 is then used to produce values for the distance between each plaque volume and the relevant ostium location, as indicated at step 138, and a plaque score is produced, for example using the above equation (2), equation (3), equation (3) or equation (4) above, as indicated at step 140.

It will be understood that using the system 80 shown in FIG. 7, a user is able to obtain a plaque score based on contrast CCTA data that is specific to a selected type of plaque volume and indicative of the distribution of the specific type of plaque across the patient's coronary arteries by applying appropriate filtering characteristics using the plaque filter 110. For example, the user may select calcified plaque so that the plaque score corresponds to the calcified plaque score produced by the system shown in FIG. 1, or the user may select vulnerable plaque of defined HU density so as to obtain a plaque score indicative of how dispersed or focused defined non-calcified plaque is for the patient.

### EXAMPLE

In an example, 762 patients were evaluated in relation to risk of major adverse cardiovascular events (MACE).

The cardiovascular event risk of a patient can be characterised in terms of the likelihood of MACE. Historical data associated with 762 patients was evaluated and an estimate made as to how much risk each patient was exposed to when a CT scan for the patient was taken.

Plaque scores in accordance with the embodiments were also determined for the 762 patients based on the patent CT scans and hazard rations for the patients compared with hazard ratios for CAD-RADS ratings. The comparison showed that the present plaque scores have higher hazard ratios than CAD-RADS, and therefore they are better predictors of MACE than CAD-RADS.

In the claims that follow and in the preceding description of the embodiments, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

It is to be understood that, if any prior art publication is referred to herein, such reference does not constitute an admission that the publication forms a part of the common general knowledge in the art, in Australia or any other country.

Modifications and variations as would be apparent to a skilled addressee are deemed to be within the scope of the embodiments.

What is claimed is:

1. A method of automatically determining a plaque score for coronary arteries of a patient, the method comprising:
receiving cardiac CT data indicative of a cardiac CT scan carried out on the patient;
analysing the cardiac CT data to identify plaque volumes to be included in the plaque score, the plaque volumes located on the coronary arteries;
determining locations of the identified plaque volumes on the coronary arteries;
applying machine learning to the cardiac CT data to identify a plurality of primary key points on the coronary arteries;
determining a distance between each identified plaque volume and an associated primary key point; and
determining a plaque score based on a heart dimension value and, for each identified plaque volume, the determined distance from the identified plaque volume to the associated primary key point.

2. A method as claimed in claim 1, wherein the locations of the identified plaque volumes on the coronary arteries are determined using machine learning.

3. A method as claimed in claim 1, wherein the key points include at least one primary key point corresponding to an ostium location at which a coronary artery connects to a patient's aorta.

4. A method as claimed in claim 3, wherein the key points include at least one secondary key point corresponding to a location on a coronary artery associated with a coronary artery branch location.

5. A method as claimed in claim 3, wherein:
if an identified plaque volume is on a coronary artery that connects directly to the aorta at an ostium, then the method comprises determining the distance by determining a straight line distance from the plaque volume to a primary key point associated with the ostium; and
if an identified plaque volume is on a coronary artery that connects indirectly to the aorta at an ostium through a branch, then the method comprises determining the distance by determining a straight line distance from the plaque volume to a secondary key point associated with the branch plus a straight line distance from the secondary key point to a primary key point associated with the ostium.

6. A method as claimed in claim 1, wherein the distance is determined from a centroid of an identified plaque volume.

7. A method as claimed in claim 1, wherein the heart dimension value includes a maximum heart diameter value.

8. A method as claimed in claim 1, wherein the plaque score is determined based on volume magnitude of the plaque volumes.

9. A method as claimed in claim 1, wherein the cardiac CT data is non-contrast CT data and the plaque volumes are calcified volumes.

10. A method as claimed in claim 9, wherein the plaque score is determined using the following equation:
$$D = {\sum }_{i = 1}^{N} {s}_{i} {w}_{i} \frac{H}{{d}_{i}}$$
where N is the total number of calcified plaque volumes with Hounsfield Units (HU) >=130; s is the volume magnitude of each calcified plaque volume in mm³; w is a weighting factor of the calcified plaque volume's maximum attenuation; d is the straight-line distance from the calcified plaque volume's centroid to the ostium (via the bifurcation point if the lesion is located beyond the left main artery) in mm; and H is the maximum heart diameter measured along a short axis of the segmented heart region in an axial view in mm;
wherein the weighting factor w is given by:
$$w ( {X}_{i} ) = \{ \begin{matrix}0 & if max ( {X}_{i} ) < 130 \\ 1 & if 130 <= max ( {X}_{i} ) <= 199 \\ 2 & if 200 <= max ( {X}_{i} ) <= 299 \\ 3 & if 300 <= max ( {X}_{i} ) <= 399 \\ 4 & if 400 <= max ( {X}_{i} ) <= 999 \\ 1 & if 1000 <= max ( {X}_{i} )\end{matrix}$$

11. A method as claimed in claim 1, wherein the cardiac CT data is contrast CT data and the plaque volumes include non-calcified volumes.

12. A method as claimed in claim 11, comprising filtering CT data using defined filtering characteristics to identify the plaque volumes to be included in the plaque score.

13. A method as claimed in claim 12, wherein the defined filtering characteristics include HU values, and/or plaque volume characteristics, and/or plaque composition.

14. A method as claimed in claim 11, wherein the plaque score is determined based on a stenosis severity parameter; and/or based on an amount of high risk plaque features, wherein each high risk plaque feature has an associated scaling factor.

15. A method as claimed in claim 11, wherein the plaque score is determined using the following equation:
$$D = {\sum }_{i = 1}^{N} ( {s}_{i} + {p}_{i} ) \frac{V}{{d}_{i}} {\sum }_{j = 1}^{3} {α}_{j} {v}_{i , j}$$
where N is the total number of plaque volumes; s is stenosis percentage/100; p is the number of high risk plaque features; V is the left ventricle volume magnitude in mm³; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque,
or using the following equation:
$$D = {\sum }_{i = 1}^{N} ( {s}_{i} + {p}_{i} ) \frac{V}{{d}_{i}} {\sum }_{j = 1}^{3} {α}_{j} {v}_{i , j}$$
where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque,
or using the following equation:
$$D = {\sum }_{i = 1}^{N} ( {s}_{i} + {p}_{i} ) \frac{V}{{d}_{i}}$$
where N is the total number of plaque volumes; s is stenosis percentage/100; p is the number of high risk plaque features; V is the left ventricle volume magnitude in mm³; and d is the straight-line distance from the plaque volume's centroid to the ostium (via the bifurcation point if the lesion is located beyond the left main artery) in mm,
or using the following equation:
$$D = {\sum }_{i = 1}^{N} ( {s}_{i} + {p}_{i} ) \frac{V}{{d}_{i}}$$
where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; and d is the straight-line distance from the plaque volume's centroid to the ostium (via the bifurcation point if the lesion is located beyond the left main artery) in mm.

16. A system for automatically determining a plaque score for coronary arteries of a patient, the system comprising:
a plaque volume identifier arranged to receive cardiac CT data indicative of a cardiac CT scan carried out on a patient, analyse the cardiac CT data to identify plaque volumes located on the coronary arteries to be included in the plaque score, and determine locations of the identified plaque volumes on the coronary arteries;
a key point determiner arranged to apply machine learning to the cardiac CT data to identify a plurality of primary key points on the coronary arteries;
a key point distance determiner arranged to determine a distance between each identified plaque volume and an associated primary key point; and
a plaque score calculator arranged to determine a plaque score based on a heart dimension value and, for each identified plaque volume, the determined distance from the identified plaque volume to the associated primary key point.

17. A system as claimed in claim 16, wherein the key points include at least one primary key point corresponding to an ostium location at which a coronary artery connects to a patient's aorta.

18. A system as claimed in claim 16, wherein the key points include at least one secondary key point corresponding to a location on a coronary artery associated with a coronary artery branch location.

19. A system as claimed in claim 16, wherein:
if an identified plaque volume is on a coronary artery that connects directly to the aorta at an ostium, then the method comprises determining the distance by determining a straight line distance from the plaque volume to a primary key point associated with the ostium, and
if an identified plaque volume is on a coronary artery that connects indirectly to the aorta at an ostium through a branch, then the method comprises determining the distance by determining a straight line distance from the plaque volume to a secondary key point associated with the branch plus a straight line distance from the secondary key point to a primary key point associated with the ostium.

20. A system as claimed in claim 16, wherein the plaque score is determined based on volume magnitude of the plaque volumes.

21. A system as claimed in claim 16, wherein the cardiac CT data is non-contrast CT data and the plaque volumes are calcified volumes.

22. A system as claimed in claim 16, wherein the plaque score is determined using the following equation:
$$D = {\sum }_{i = 1}^{N} {s}_{i} {w}_{i} \frac{H}{{d}_{i}}$$
where N is the total number of calcified plaque volumes with Hounsfield Units (HU) >=130; s is the volume magnitude of each calcified plaque volume in mm³; w is a weighting factor of the calcified plaque volume's maximum attenuation; d is the straight-line distance from the calcified plaque volume's centroid to the ostium (via the bifurcation point if the lesion is located beyond the left main artery) in mm; and H is the maximum heart diameter measured along a short axis of the segmented heart region in an axial view in mm,
wherein the weighting factor w is given by:
$$w ( {X}_{i} ) = \{ \begin{matrix}0 & if max ( {X}_{i} ) < 130 \\ 1 & if 130 <= max ( {X}_{i} ) <= 199 \\ 2 & if 200 <= max ( {X}_{i} ) <= 299 \\ 3 & if 300 <= max ( {X}_{i} ) <= 399 \\ 4 & if 400 <= max ( {X}_{i} ) <= 999 \\ 1 & if 1000 <= max ( {X}_{i} )\end{matrix}$$

23. A system as claimed in claim 16, wherein the cardiac CT data is contrast CT data and the plaque volumes include non-calcified volumes.

24. A system as claimed in claim 23, comprising a plaque filter for filtering CT data using defined filtering characteristics to identify the plaque volumes to be included in the plaque score.

25. A system as claimed in claim 24, wherein the defined filtering characteristics include HU values, and/or plaque volume characteristics, and/or plaque composition.

26. A system as claimed in claim 23, wherein the plaque score is determined based on a stenosis severity parameter; and/or based on an amount of high risk plaque features, wherein each high risk plaque feature has an associated scaling factor.

27. A system as claimed in claim 23, wherein the plaque score is determined using the following equation:
$$D = {\sum }_{i = 1}^{N} ( {s}_{i} + {p}_{i} ) \frac{V}{{d}_{i}} {\sum }_{j = 1}^{3} {α}_{j} {v}_{i , j}$$
where N is the total number of plaque volumes; s is stenosis percentage/100; p is the number of high risk plaque features; V is the left ventricle volume magnitude in mm³; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque,
or using the following equation:
$$D = {\sum }_{i = 1}^{N} ( {s}_{i} + {p}_{i} ) \frac{V}{{d}_{i}} {\sum }_{j = 1}^{3} {α}_{j} {v}_{i , j}$$
where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; d is the straight-line distance from the plaque volume's centroid to the ostium (via the bifurcation point if the lesion is located beyond the left main artery) in mm; v is the volume magnitude of a plaque volume in mm³; and α is a constant scaling factor that may be for example 0.1 for calcified plaque, 0.3 for non-calcified plaque and 0.6 for low attenuation plaque,
or using the following equation:
$$D = {\sum }_{i = 1}^{N} ( {s}_{i} + {p}_{i} ) \frac{V}{{d}_{i}}$$
where N is the total number of plaque volumes; s is stenosis percentage/100; p is the number of high risk plaque features; V is the left ventricle volume magnitude in mm³; and d is the straight-line distance from the plaque volume's centroid to the ostium (via the bifurcation point if the lesion is located beyond the left main artery) in mm,
or using the following equation:
$$D = {\sum }_{i = 1}^{N} ( {s}_{i} + {p}_{i} ) \frac{V}{{d}_{i}}$$
where N is the total number of plaque volumes; s=1 if stenosis is greater than or equal to 50% in the LM coronary artery or is greater than or equal to 70% in other vessels, and otherwise s=0; p is the number of high risk plaque features; V is the left ventricle volume magnitude in mL; and d is the straight-line distance from the plaque volume's centroid to the ostium (via the bifurcation point if the lesion is located beyond the left main artery) in mm.
